(19) United States
(12) Patent Application Publication
(10) Pub. No.: US 2025/0066432 A1
(43) Pub. Date: Feb. 27, 2025

(54) RECOMBINANT TYPE I ALLERGEN OF ARTEMISIA VULGARIS POLLEN, AND PREPARATION METHOD AND USE THEREOF

(71) Applicant: ZONHON BIOPHARMA INSTITUTE, INC., Changzhou (CN)

(72) Inventors: Bruce Yong MA, Changzhou (CN); Baixue ZHAO, Changzhou (CN); Hui GAO, Changzhou (CN); Yu ZHUANG, Changzhou (CN); Qin WANG, Changzhou (CN)

(73) Assignee: ZONHON BIOPHARMA INSTITUTE, INC., Changzhou (CN)

(21) Appl. No.: 18/725,846

(22) PCT Filed: Dec. 30, 2022
(86) PCT No.: PCT/CN2022/144040

§ 371 Date: Jul. 1, 2024

(30) Foreign Application Priority Data
Dec. 30, 2021 (CN) 202111658039.9

(51) Int. Cl. C07K 14/415; C07K 1/36; C12N 15/81

(52) U.S. Cl. CPC C07K 14/415; C07K 1/36; C12N 15/815

(57) ABSTRACT

A recombinant type I allergen of Artemisia vulgaris pollen (Art v 1), a coding gene, an expression method, and a purification method thereof are provided. By different combinations of codon optimization, different signal peptides, expression vectors and strains, the Art v 1 obtained reaches the expression level of more than 200 mg/L, with a purity of more than 99%, and the activity of the Art v 1 is equivalent to that of a native protein, and the Art v 1 can be used for the desensitization immunotherapy and diagnosis of Artemisia pollen allergy.

### CROSS REFERENCE TO THE RELATED APPLICATIONS

This application is the national phase entry of International Application No. PCT/CN2022/144040, filed on Dec. 30, 2022, which is based upon and claims priority to Chinese Patent Application No. 202111658039.9, filed on Dec. 30, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBNJIP145-Sequence-Listing.xml, created on Jul. 1, 2024, and is 20,786 bytes in size.

### TECHNICAL FIELD

The present disclosure belongs to the technical field of biological medicines and relates to a recombinant type I allergen of Artemisia vulgaris pollen, the characterization and activity of which are consistent with those of a native allergen, and a preparation method and a use thereof.

### BACKGROUND

Pollen is one of the main triggers of seasonal allergy. Unlike food allergy, pollen allergy is transmitted through the air and is often difficult to avoid. Pollen allergy can induce a series of allergic reactions such as rhinitis, dermatitis and asthma, which seriously affect the quality of life of patients. Pollen allergy affects about 7% of adults and 9% of children in the United States (NIAID, National Institute of Allergy and Infectious Diseases), and prevalence is estimated to be as high as 40% in Europe (G.D'Amato, 2007). In recent years, with the increasing area of green land and the “grain for green” program in China, the incidence of pollen allergy has been increasing year by year, and in the high incidence area can reach 5%.

The Artemisia pollen is one of the important allergens that cause hay fever in summer and autumn. The results of 215210 allergen specific IgE tests in China from 2008 to 2010 showed that Artemisia pollen had the highest positive rate among inhaled allergens. Qiongliang Yang et al. showed in 2015 that Artemisia pollen was the most important allergen in northern China.

Artemisia L. is one of the most abundant genera in the Asteraceae family. There are more than 300 Artemisia species in the world, which are widely distributed in temperate, temperate cold and subtropical regions of the Northern Hemisphere. Artemisia annus and Mugwort are common pollen-sensitized plants. Among them, Artemisia annus, which can be used to extract artemisinin, is one of the most common Artemisia plants in China, and it is also the earliest Artemisia allergic plant studied in China. Artemisia vulgaris, also known as mugwort, is widely distributed in China, Mongolia, Russia, Europe, the United States, Canada and other places. It is one of the most deeply studied pollen allergens. Other major sensitized Artemisia species include Artemisia sieversiana, Artemisia capillaris, Artemisia lavabdykufikua, and Artemisia desertorum, Artemisia argyi, etc. The main allergenic proteins of different Artemisia pollen are type I and type III allergens. Type I allergen belongs to the defensin-like protein family with a molecular weight of about 12 kD. Type III allergens belong to non-specific lipid transport protein (nsLTP), which has relatively high variability in different Artemisia pollen.

The World Health Organization (WHO) proposes a “four-in-one” approach to allergic diseases: allergen avoidance, symptomatic drug treatment, specific immunotherapy, and physician and patient education. Among them, avoiding exposure to allergens refers to doing a good job in environmental prevention and control on the basis of identifying allergens and avoiding exposure to allergens as far as possible. Avoiding allergens in the treatment of allergic diseases can not only reduce the incidence of allergy, but also improve the efficacy of drugs and help patients desensitize faster. Specific immunotherapy, namely desensitization therapy, is the only “cause-specific” therapy that may affect the natural course of allergic diseases and change the immune response mechanism. It uses gradually increasing doses of allergens to improve the patient's tolerance to the allergen, reduce the symptoms caused by exposure to the allergen, and eventually achieve tolerance or even immune tolerance.

In 2021, Zhejiang Wolwo pharma's desensitization drug, Artemisia annus pollen allergen sublingual drops for the treatment of allergic rhinitis caused by Artemisia annus/Mugwort pollen, was approved for marketing. The main ingredient is allergen protein extract from Artemisia annus pollen, and its patent CN101905022A states that “As raw material, Artemisia pollen was defatted, extracted and concentrated to prepare Artemisia pollen allergen extract. However, as a result of the limitation of raw material sources and production methods, allergen extracts inevitably exist quality problems, such as presence of undefined non-allergic substances, pollutants and high variability in allergen content and biological activity (Valenta R, et al. Allergen Extracts for in vivo diagnosis and treatment of allergy: is there a future [J]. Journal of allergy &Clinical immunology in practice, 2018). The EAACI Guidelines on allergen immunotherapy: allergic rhinoconjunctivitis (2018) issued by the European Academy of Allergy and Clinical Immunology also states that: There are many potential disadvantages of mixed allergens, including dilution effects, potential allergen degradation due to the enzymatic activity of some allergens, and difficulties in adequately demonstrating the efficacy of allergen combinations. The standardized desensitizing drugs approved for marketing by EMA, HMA, and FDA are basically limited to the major allergenic proteins. For Example, ODACTRA for house dust mite allergy contains major allergenic proteins Der p 1, Der p 2, Der f 1 and Der f 2. GRAZAX for timothy allergy contains Phl p 5. RAGWIZAX for ragweed allergy contains the major allergenic protein Amb a 1. On the other hand, the use of natural extracts for allergen diagnosis has the problems of low sensitivity and specificity, and it is not possible to determine the reaction degree to allergen components, which may easyly lead to misdiagnosis.

At present, there is no recombinant Artemisia pollen allergen protein as drug on the market or in clinical trials.

### SUMMARY

The present disclosure provides a recombinant Artemisia vulgaris pollen allergen with clear main allergenic protein, so as to improve the controllability of product quality, ensure the precision of desensitization immunotherapy drugs for Artemisia pollen allergy and the accuracy of allergy diagnosis, and lay the foundation for the medical use of recombinant Artemisia pollen allergen.

An objective of the present disclosure is to provide a protein for treating Artemisia pollen allergy, which is a recombinant Art v 1 protein. Art v 1 is type I allergen protein of Artemisia vulgaris and is major allergenic protein. It is a glycoprotein composed of the N-terminal defensin domain and the C-terminal hydroxyproline-rich part, belonging to the defensin-like protein family. Numerous researchs have shown that the type I allergen proteins of different Artemisia species are all defensin proteins with highly conserved sequences. The binding ability of each subtype to sIgE antibody is similar, and its immune activity is mainly determined by the N-terminal defensin domain. The amino acid sequence, disulfide bond and molecular weight of the recombinant Art v 1 protein is completely consistent with the natural protein, and it has similar biological activity to the native protein.

Preferably, the amino acid sequence of this Art v 1 protein is set forth as SEQ ID NO: 4.

The amino acid sequence, molecular weight, amino acid coverage and disulfide bond of the recombinant Art v 1 protein in the present disclosure are completely consistent with the native Art v 1 protein, and it has similar immunological activity with native Art v 1. Compared with the natural Artemiha pollen extract, variation in content and activity between batches is avoided, the process and quality is more stable and controllable, the degradation of main allergens and other allergic reactions caused by the interaction of other components in natural pollen is avoided, so as to meet the requirements of safe, effective and controllable quality of modern biological products. It can be used in the treatment and diagnosis of Artemisia pollen allergy such as allergic rhinitis and asthma, and improve the accuracy of Artemisia pollen desensitization immunotherapy and the accuracy of Artemisia pollen allergy diagnosis.

A further objective of the present disclosure is to provide DNA sequence encoding the Art v 1 protein with base sequence as set forth in SEQ ID NO: 13. This sequence is codon optimized for the Pichia pastoris expression system, which is more conducive to the expression of Art v 1 in Pichia pastoris.

Another purpose of the present disclosure is to provide secretory signal peptide design that is beneficial to the expression of Art v 1 protein in Pichia pastoris expression system, which not only improves the expression of Art v 1 protein, but also the molecular characterization of the obtained recombinant Art v 1 protein is completely consistent with that of the native protein. The signal peptide is the yeast α-factor signal peptide, the melanomycin signal peptide, the acid phosphatase signal peptide (PHO), the Saccharomyces cerevisiae signal peptide (SUC2) and the Art v 1 protein wild type signal peptide, preferably, the signal peptide is the α-factor signal peptide (SEQ ID NO: 11) and wild type signal peptide (SEQ ID NO: 12). The inventors found that different secretory signal peptides had significant effect on the uniformity and expression of the recombinant Art v 1 protein, and the preferred signal peptide was more conducive to the correct and efficient expression. The recombinant Art v 1 obtained was not only highly expressed, but also was completely consistent with native Art v 1 in the primary structure, molecular weight, amino acid coverage and disulfide bond.

A further objective of the present disclosure is to provide a vector containing the above gene encoding Art v 1, preferably, the vector is pAO815, pPIC9, pPIC9K, pPIC3.5, pIC3.5K, pPICZαA, B, C or pGAPZαA, B, C. More preferably, the vector is pPICZ A or pGAPZ A.

A further objective of the present disclosure is to provide a Pichia pastoris strain comprising the vector described above. Preferably, the Pichia pastoris strain is SMD1168, GS115, KM71, X33 or KM71H, more preferably is the KM71 or X33 strain.

The gene coding recombinant protein of the present disclosure is more conducive to Pichia pastoris expression. The inventors found that different combinations of signal peptides, expression vectors, and host bacterial have a significant impact on the yield of recombinant Art v 1. The best combination has a yield of 210 mg/L. The purified recombinant Art v 1 protein has the same amino acid sequence, disulfide bond and molecular weight as the native protein, and showed similar immune response activity in vitro with specific antibodies in the serum of allergic patients as the natural protein.

A further objective of the present disclosure is to provide expression method of Art v 1 protein, comprising the steps of:
A. When the common pPICZ series or pGAPZ series are used as vectors, the exogenous gene expression cassette does not contain the signal peptide, so it can be used to construct the Art v 1 expression cassette containing wild type signal peptide. Specifically, the artificial sequence of Art v 1 containing wild type signal peptide (designed with a start codon and a stop codon) is cloned to the polyclonal sites (such as EcoRI and NotI) of the corresponding vector, so that the open reading frame is located downstream of the promoter and upstream of the terminator to construct a recombinant expression vector.

When the common pPICZα series or pGAPZα series are used as vectors, the exogenous gene expression cassette contains α-factor signal peptide and signal peptide cleavage site: Kex2 (amino acid sequence is KR) and Ste 13 (amino acid sequence is EAEA, SEQ ID NO: 15). The Art v 1 target gene could be cloned to downstream of the Kex2 sequence (the α-factor signal peptide is removed by Kex2 protease) or downstream of the Ste 13 sequence (the α-factor signal peptide could be removed by Kex2 and/or Ste 13 protease at the same time). Taking the pPICZαA vector as an Example, if the Art v 1 gene sequence is located after Kex2, the target gene can be cloned between XhoI and NotI sites. If the Art v 1 gene sequence is located after Ste 13, the target gene can be cloned between EcoRI and NotI sites. The recombinant expression vector expressing Art v 1 using α-factor signal peptide is constructed.
B. The vector of step A is linearized and transferred into Pichia pastoris strain, and the clone with high expression is screened under the pressure of antibiotic, and the expression level is further verified by cultivation or purification with suitable condition.
C. Recovery of purified proteins.

Such vectors are preferably pPICZα A or pGAPZα A.

The Pichia pastoris strains described above are preferably KM71 or X33 strains.

A further objective of the present disclosure is to provide purification method of recombinant Art v 1 protein as follows:
A. Art v 1 fermentation broth is centrifugated at low temperature and high-speed, the supernatant is collected and concentrated by ultrafiltration with 3KD membrane package, replaced by pH7.0 25 mM PB buffer, and filtered by 0.45 μm filter membrane.
B. In the first step of cation chromatography, the column is equilibrated with equilibrium buffer, and then the fermentation liquid obtained in step A is passed through the separation filler by the purification system, then the elution buffer is used for gradient elution, and the elution peak is collected; Equilibration buffer is 25 mM PB, pH7.0, and elution buffer is 25 mM PB, 1.0M NaCl, pH7.0.
C. In the second step, the Art v 1 protein peak collected in B is diluted with equilibrium buffer, and the chromatographic column is equilibrated by equilibrium buffer. The diluted Art v 1 protein solution is loaded onto the hydrophobic chromatographic packing and collect the elution peak. Equilibration buffer is 1.0M (NH₄)₂SO₄, 25 mM PB, pH7.0, and elution buffer is 25 mM PB, pH7.0.
D. In the third step, the target protein peak collected in C is ultrafiltered and replaced with pH7.0 25 mM PB buffer. Art v 1 protein stock solution is obtained after filtration and bacteria removing.

After optimizing the cultivation process and purification method, the recombinant Art v 1 prepared by the present disclosure meets the requirements of recombinant DNA products for human use in terms of purity, impurity residue, molecular characterization, etc. The SEC-HPLC purity is >99%, and the expression level reaches 210 mg/L. It has the same amino acid sequence, disulfide bond and molecular weight as the native protein, and its in vitro immune reaction activity with specific antibodies in the serum of allergic patients is equivalent to the native protein. It has good potential for medicinal use. Compared with naturally extracted allergen products, recombinant allergen molecules have many advantages, such as variation in content and activity between batches is avoided; the process and quality is more stable and controllable, and avoid the degradation of main allergens and avoid other allergic reactions caused by the interaction of other components in native pollen, meet the needs of safe, effective and controllable quality of modern biological products; In addition, allergy diagnostic kits using recombinant allergen protein can accurately identify the allergen protein that triggers the body reaction.

### BRIEF DESCRIPTION OF THE DRAWINGS

FIG. 1 represented comparison of the Art v 1 gene sequence before and after optimization.

The unoptimized sequence was the nucleotide sequence of the native Art v 1 gene. Art v 1-01 was the first optimized nucleotide sequence, and Art v 1-02 was the second optimized nucleotide sequence.

FIGS. 2A, 2B, and 2C showed the average GC base content distribution of Art v 1 gene before and after codon optimization in the Pichia pastoris expression system.

FIG. 2A showed that the average GC base content of native Art v 1 gene in Pichia pastoris expression system was 50.71%. FIG. 2B showed that the average GC base content of Art v 1-01 codon in Pichia pastoris expression system was 55.48%. FIG. 2C indicated that the average GC base content of the Art v 1-02 codon in the Pichia pastoris expression system was 56.31%.

FIG. 3 showed the agarose gel electrophoresis of the PCR products of the Art v 1-01 and Art v 1-02 genes (containing wild type signal peptide) after codon optimization. Lane 1 was a 200 bp DNA Ladder. Lane 2 was the PCR product of Art v 1-01 gene. Lane 3 was the PCR product of the Art v 1-02 gene.

FIG. 4 showed the agarose gel electrophoresis of the PCR products of the Art v 1-01 and Art v 1-02 genes (without wild type signal peptide) after codon optimization. Lane 1 was the PCR product of Art v 1-01 gene. Lane 2 was a 200 bp DNA Ladder; Lane 3 was the PCR product of the Art v 1-02 gene.

FIGS. 5A-5B showed the expression identification of Art v 1-01, 02 genes in four pPIC engineered bacteria.

Among them, FIG. 5A showed the SDS-PAGE gel electrophoresis of bacterial supernatant after 72 hours expression of pPICZ-Art v 1-01 engineered strain. Lane 1 was 10-94 kD non-pre-stained protein marker. Lane 2-9 was the culture supernatant of each positive monoclonal engineering strain with Art v 1-01 gene screened by Zeocin.

FIG. 5B showed the SDS-PAGE gel electrophoresis of bacterial supernatant after 72 hours expression of pPICZα-Art v 1-02 engineered strain. Lane 1 was 10-94 kD non-pre-stained protein marker. Lane 2-10 was the culture supernatant of each positive monoclonal engineering strain with Art v 1-02 gene screened by Zeocin.

FIGS. 6A-6B showed the expression identification of Art v 1-01,02 genes in four pGAP engineered bacteria.

FIG. 6A showed the SDS-PAGE gel electrophoresis of the bacterial supernatant after 48 hours expression of pGAPZ-Art v 1-01 engineered strain. Lane 1 was 10-94 kD non-pre-stained protein marker. Lane 2-10 was the culture supernatant of the monoclonal engineering strain with Art v 1-01 gene screened by Zeocin.

FIG. 6B showed the SDS-PAGE gel electrophoresis of bacterial supernatant after 48 hours expression of pGAPZα-Art v 1-02 engineered strain. Lane 1 was 10-94 kD non-pre-stained protein marker. Lane 2-10 was the culture supernatant of the monoclonal engineering strain with Art v 1-02 gene screened by Zeocin.

FIGS. 7A-7B showed the purification chromatogram and electrophoretic identification of recombinant Art v 1 fermentation broth in the first cation chromatography.

FIG. 7A showed the purification chromatogram of recombinant Art v 1 fermentation broth supernatant in the first cation chromatography. There are three elution peaks.

FIG. 7B showed the electrophoresis of recombinant Art v 1 fermentation supernatant after the first step of cation chromatography. Lane 1 was 10-94 kD non-prestained protein Marker; Lane 2 was the sample of Art v 1 fermentation broth before purification. Lane 3 was the breakthrough sample of Art v 1 fermentation broth in the first cation chromatography step. Lane 4 was the elution peak 1 of Art v 1 fermentation broth purified by the first cation chromatography. Lane 5 was the elution peak 2 of Art v 1 fermentation broth purified by the first cation chromatography. Lane 6 was the elution peak 3 of Art v 1 fermentation broth purified by the first cation chromatography.

FIGS. 8A-8B showed the purification chromatogram and electrophoretic identification of recombinant Art v 1 protein in the second hydrophobic chromatography.

Among them, FIG. 8A showed the purification chromatogram of recombinant Art v 1 protein in the second hydrophobic chromatography, with only one elution peak.

FIG. 8B showed the electrophoresis of the recombinant Art v 1 protein after the second hydrophobic chromatography. Lane 1 was 10-94 kD non-prestained protein marker. Lane 2 was the elution peak 1 of recombinant Art v 1 protein purified by the second hydrophobic chromatography.

FIGS. 9A-9B showed the purification chromatogram and electrophoretic identification of native Art v 1 protein.

Among them, FIG. 9A showed the purification chromatogram of native Art v 1 protein in cation chromatography, with three elution peaks.

FIG. 9B showed the electrophoresis of native Art v 1 protein after the cation chromatography. Lane 1 was 10-94 kD non-prestained protein marker; Lane 2 was elution peak 1; Lane 3 was elution peak 2. Lane 4 was elution peak 3.

FIG. 10 showed the results of peptide coverage detection of recombinant Art v 1 protein.

FIGS. 11A-11B showed the disulfide bond identification of Art v 1 protein. Among them, FIG. 11A showed the disulfide bond identification of the recombinant Art v 1 protein, and FIG. 11B showed the disulfide bond identification of the native Art v 1 protein.

FIG. 12 showed the results of SEC-HPLC purity determination of recombinant Art v 1 protein.

FIG. 13 showed the inhibition curves of the recombinant Art v 1 protein and the native Art v 1 protein against IgE antibodies in positive sera

### DETAILED DESCRIPTION OF THE EMBODIMENTS

The present disclosure was further described below in connection with specific examples, and it was understood that the examples were cited only to illustrate the present disclosure and not to limit the scope of the present disclosure.

### Example 1: Codon Optimization of the Art v 1 Gene

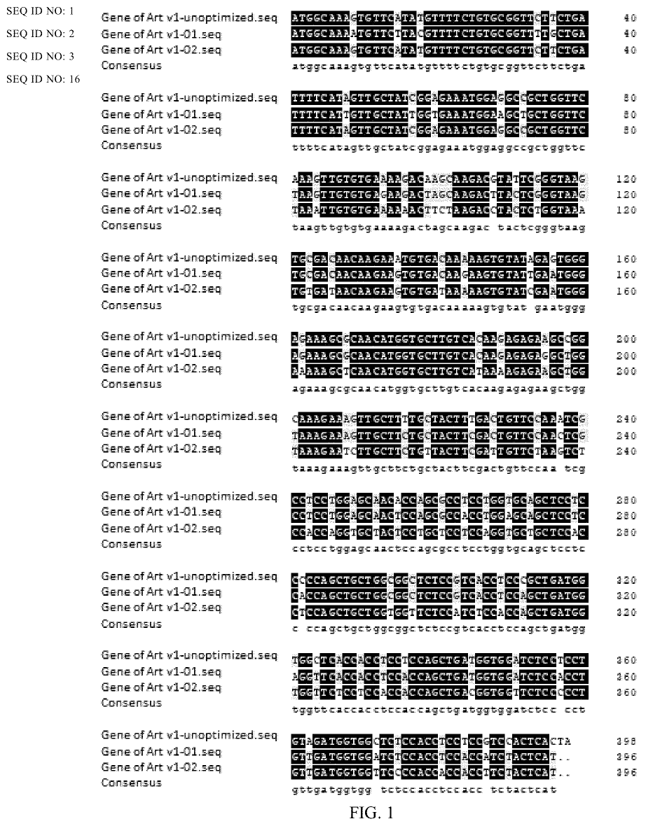

According to the DNA sequence of Art v 1 published by NCBI (Genbank accession number: AF493943, containing wild type signal peptide), as set forth in SEQ ID NO: 1, the inventors optimized the codon of the gene to obtain two gene sequences containing wild type signal: Art v 1-01 and Art v 1-02, the nucleotide sequences were shown as SEQ ID NO: 2 and SEQ ID NO: 3, respectively, and the amino acid sequence was shown as SEQ ID NO: 4. Comparison of base sequences before and after codon optimization was shown in FIG. 1.

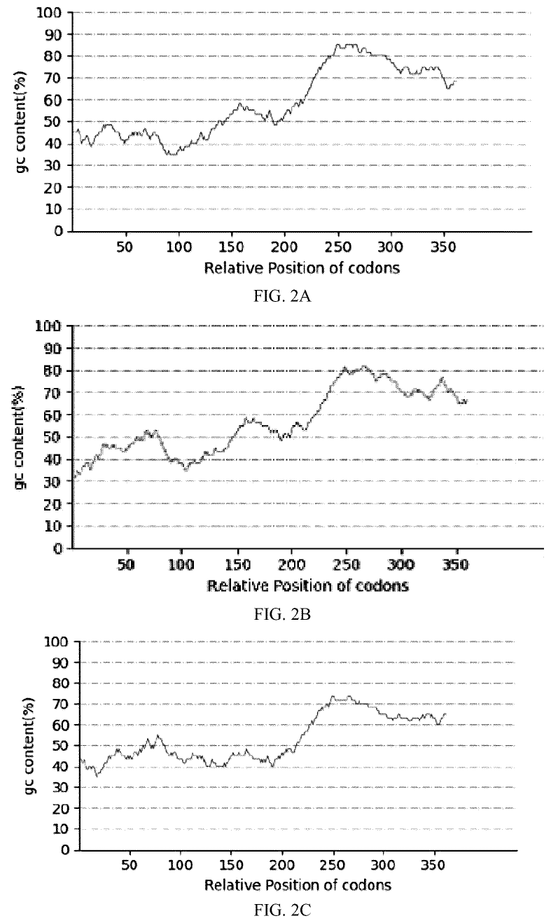

The GC content can affect the expression level of genes. The ideal GC content is 30%-70%, and GC content beyond this range will affect transcription and translation efficiency. The average GC base content of Art v 1 gene in FIG. 2A was 50.71%, and the average GC base content of Art v 1-01 after optimization in FIG. 2B was 55.48%. In FIG. 2C, the average GC base content of optimized Art v 1-02 was 56.31%. After optimization, the average GC content was increased, and there was no significant difference between Art v 1-01 and Art v 1-02.

### Example 2: Construction of Art v 1 Gene Expression Plasmid Containing Wild Type Signal Peptide

1. pPIC Expression Plasmid was Constructed

Enzyme cutting site sequence EcoR I and XhoI were introduced to the 5′ end and 3′ end of the codon-optimized Art v 1-01 and 02 genes in Example 1, and synthesize the whole gene. The synthesized gene fragment was constructed into pPICZ plasmid (provided by GenScript Biotech Corporation) to obtain long-term preservation plasmids, which were denoted as pPICZ-Art v 1-01 and pPICZ-Art v 1-02 according to different optimization methods.

2. pGAP Expression Plasmid was Constructed

pPICZ-Art v 1-01 and pPICZ-Art v 1-02 plasmids were used as templates for PCR amplification with the following primer sequences:
5′AOX primer was used as forward primer with a sequence as set forth in SEQ ID NO: 5; 3′AOX primer was used as reverse primer with a sequence as set forth in SEQ ID NO: 6.

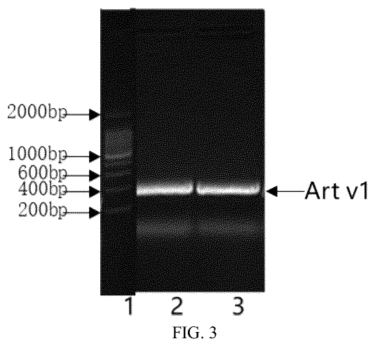

The total volume of reaction system was 50 μL, including 2.5 μL of each primers at a concentration of 10 μmol/L, 1 μL of 10 mmol/L dNTP, and 0.5 μL of 2 U/μL DNA polymerase Q5 (purchased from New England Biolabs). The reaction condition was 98° C. for 5 seconds, 55° C. for 45 seconds, and 72° C. for 30 seconds, and after 25 cycles, the products were analyzed by 1.0% agarose gel electrophoresis which showed that the product had the expected size (400 bp) (the results were shown in FIG. 3). After double digestion with Xho I(R0146S, New England Biolabs) and EcoR I(R3101S, New England Biolabs), the products were subjected to 1% agarose electrophoresis and then purified by DNA gel recovery kit (DP214, Beijing Tiangen Biochemical Technology Co., LTD.).

The purified product was was ligated into pGAPZ A plasmid (purchased from Invitrogen) with T4 ligase (M0202S, purchased from New England Biolabs) and transformed into DH5α competent cells (CB101, purchased from Beijing Tianggen Biochemical Technology Co., LTD.). Culture overnight at 37° C. in LB solid medium containing bleomycin (purchased from Invitrogen). The next day, the positive clones were selected for sequencing, which were completely consistent with the expected sequence. The expression plasmids with optimized Art v 1 codon were obtained, which were denoted as pGAPZ-Art v 1-01 and pGAPZ-Art v 1-02.

### Example 3: Construction of Art v 1 Gene Expression Plasmid with Yeast α-Factor Signal Peptide

The pPICZ-Art v 1-01 plasmid was used as template and PCR amplification was performed to obtain the Art v 1-01 gene without signal peptide, as set forth in SEQ ID NO: 13. Primer sequences was used as follows: the forward primer was SEQ ID NO: 7; the reverse primer was SEQ ID NO: 8.

The pPICZ-Art v 1-02 plasmid was used as template and PCR amplification was performed to obtain the Art v 1-02 gene without the signal peptide as set forth in SEQ ID NO: 14. Primer sequences was used as follows: the forward primer was SEQ ID NO: 9; The reverse primer was SEQ ID NO: 10.

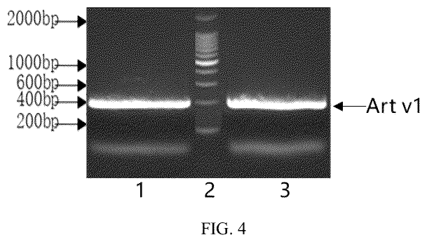

The total volume of reaction system was 50 μL, including 2.5 μL of each primers at a concentration of 10 μmol/L, 1 μL of 10 mmol/L dNTP, and 0.5 μL of 2 U/μL DNA polymerase Q5 (purchased from New England Biolabs). The reaction condition was 98° C. for 5 seconds, 55° C. for 45 seconds, and 72° C. for 30 seconds, and after 25 cycles, the products were analyzed by 1.0% agarose gel electrophoresis, which showed that the product had the expected size (400 bp) (the results were shown in FIG. 4). After double digestion with Xho I(R0146S, purchased from New England Biolabs) and Xba I(R01445S, purchased from New England Biolabs), the gene products obtained were subjected to 1% agarose electrophoresis, and then purified by the DNA gel recovery kit (DP214, Beijing Tiangen Biochemical Technology Co., LTD.).

1. pPICZα Expression Plasmid was Constructed

The purified product was ligated into pPICZαA plasmid (purchased from Invitrogen) with T4 ligase (M0202S, purchased from New England Biolabs) and transformed into DH5α competent cells (CB101, purchased from Beijing Tianggen Biochemical Technology Co., LTD.). Culture overnight at 37° C. in LB solid medium containing bleomycin (purchased from Invitrogen). The next day, the positive clones were selected for sequencing, which were completely consistent with the expected sequence. The expression plasmids with optimized Art v 1 codon were obtained, which were recorded as pPICZα-Art v 1-01 and pPICZα-Art v 1-02.

2. The pGAPZα Expression Plasmid was Constructed

The purified product was ligated into pGAPZαA plasmid (purchased from Invitrogen) with T4 ligase (M0202S, purchased from New England Biolabs) and transformed into DH5α competent cells (CB101, purchased from Beijing Tianggen Biochemical Technology Co., LTD.). Culture overnight at 37° C. in LB solid medium containing bleomycin (purchased from Invitrogen). The next day, the positive clones were selected for sequencing, which were completely consistent with the expected sequence. The expression plasmids with optimized Art v 1 codon were obtained, which were denoted as pGAPZα-Art v 1-01 and pGAPZα-Art v 1-02.

### Example 4: Art v 1 Expression Plasmid Transformation and Engineered Strains Screening

Preparation of YPDS+Zeocin solid medium: According to the description of Pichia expression vectors for constitutive expression and purification of recombinant proteins of Invitrogen, yeast extract 10 g/L, peptone 20 g/L, glucose 20 g/L, AGAR 15 g/L, sorbitol 18 g/L, and Zeocin at a final concentration of 0.1 mg/ml was included.

1, pPIC Expression Plasmid Transformation and Engineering Strain Screening

Electrocompetent cells were prepared according to the description of Easy Select Pichia Expression Kit of Invitrogen. Plasmids pPICZ-Art v 1-01, pPICZ-Art v 1-02, pPICZα-Art v 1-01 and pPICZα-Art v 1-02 obtained from Example 2 step 1 and Example 3 step 1 were digested and linearized with Sac I restriction enzyme (purchased from New England Biolabs). After ethanol precipitation, the linearized vectors were electrotransformed into Pichia pastoris X33 competent cells, coated in YPDS solid medium, and cultured at 30° C. until the transformants grew.

2, pGAP Expression Plasmid Transformation and Engineering Strain Screening

The electroconversion competent cells were prepared according to the description of Pichia expression vectors for constitutive expression and purification of recombinant proteins. Plasmids pGAPZ-Art v 1-01, pGAPZ-Art v 1-02, pGAPZα-Art v 1-01 and pGAPZα-Art v 1-02 obtained from Example 2 step 2 and Example 3 step 2 were digested and linearized with Avr II restriction enzyme (R0174S, purchased from New England Biolabs), respectively. After ethanol precipitation, the linearized vectors were electrotransformed into Pichia pastoris X33 competent cells, coated in YPDS solid medium, and cultured at 30° C. until the transformants grew.

### Example 5: Induced Expression and Identification of Art v 1 Genetic Engineered Strains 1, pPIC Clone Screening and Identification

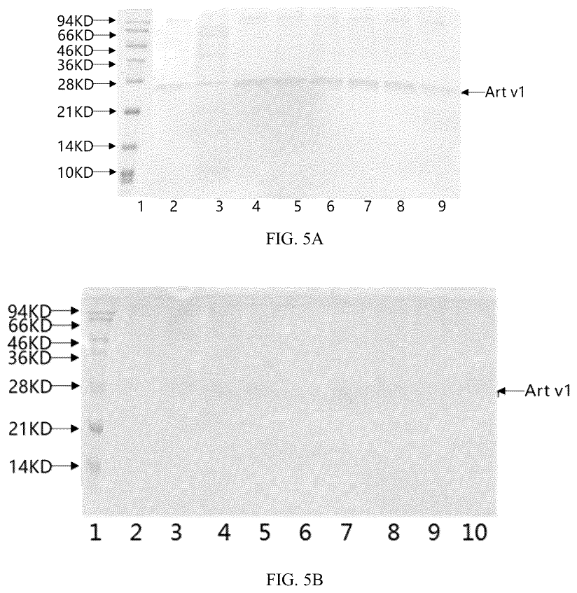

The monoclonal engineering bacteria obtained in Example 4 step 1 was selected and cultured in 5 mL BMGY medium in a 50 mL sterile centrifuge tube at 30° C. When OD600=1.0-2.0, the bacterial solution was centrifuged at 4000 rpm for 10 minutes, resuspended in BMMY medium, and induced for expression, and methanol was added every 24 hours to a final concentration of 1%. After being cultured at 220 rpm for 72 hours, centrifugated the bacterial solution, collected the supernatant, and the supernatant was analyzed by SDS-PAGE gel electrophoresis to observe the brightness of the expressed product. FIG. 5A and FIG. 5B showed the induced expression of recombinant strains with pPICZ-Art v 1-01 and pPICZα-Art v 1-02 plasmid, respectively. The expression results of strains with other construction methods were not all listed, and the expression level was shown in Table 1. The results showed that Art v 1 protein was expressed in the engineered strains with different construction methods. The strain pPICZα-Art v 1-01 had the highest expression level (no Ste 13 protease site between α-factor and target gene), reached 200 mg/L.

BMGY+zeocin medium preparation: according to description of Easy SelectPichia Expression Kit from Invitrogen, yeast extract 10 g/L, peptone 20 g/L, K₂HPO₄ 3 g/L, KH₂PO₄ 11.8 g/L, YNB 13.4 g/L, Biotin 4×10⁻⁴ g/L, glycerol 10 g/L, and Zeocin at a final concentration of 0.1 mg/ml was included.

BMMY+Zeocin medium preparation: according to description of Easy SelectPichia Expression Kit from Invitrogen, yeast extract 10 g/L, peptone 20 g/L, K₂HPO₄ 3 g/L, KH₂PO₄ 11.8 g/L, YNB13.4 g/L, Biotin 4×10⁻⁴ g/L, methanol 5 mL/L, and Zeocin at a final concentration of 0.1 mg/ml was included.

2, pGAP Clone Screening and Identification

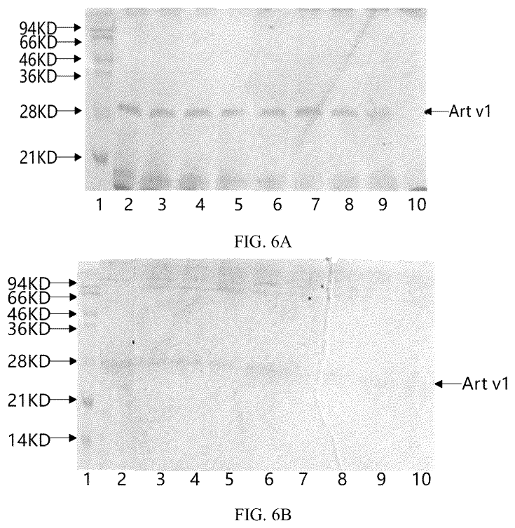

The monoclonal engineering bacteria obtained in Example 4 step 2 were selected and cultured in 5 mL YPD medium in 50 mL sterile centrifuge tube at 30° C. and 220 rpm for 48 hours. Centrifugated the bacterial solution, collected the supernatant, and the supernatant was analyzed by SDS-PAGE gel electrophoresis to observe the brightness of the expressed product. FIG. 6A and FIG. 6B showed the induced expression results of engineering strains with pGAPZ-Art v 1-01 and pGAPα-Art v 1-02 plasmid, respectively. The expression results of strains with other construction methods were not all listed, and the expression level was shown in Table 1. The results showed that Art v 1 protein was expressed in engineered strains with different construction methods. The expression level of pGAPZα-Art v 1-01 strain was the highest, reached 210 mg/L. The expression level of pGAPZα-Art v 1-02 strain was the lowest, only 30 mg/L.

Preparation of YPD+Zeocin medium: according to description of Pichia expression vectors for constitutive expression and purification of recombinant proteins from Invitrogen, yeast extract 10 g/L, peptone 20 g/L, glucose 20 g/L, and Zeocin at a final concentration of 0.1 mg/ml was included.

TABLE 1
Expression of recombinant Art v 1 with different constructs

| Genetic | | Expression | Expression |
|---|---|---|---|
| sequence | Signal peptide | system | level (mg/L) |
| Art v 1-01 | wild type signal peptide | pPICZ | 100 |
| Art v 1-02 | wild type signal peptide | pPICZ | 30 |
| Art v 1-01 | α-factor signal peptide\textcircled{1} | pPICZα | 190 |
| Art v 1-02 | α-factor signal peptide\textcircled{1} | pPICZα | 60 |
| Art v 1-01 | α-factor signal peptide\textcircled{2} | pPICZα | 200 |
| Art v 1-02 | α-factor signal peptide\textcircled{2} | pPICZα | 50 |
| Art v 1-01 | wild type signal peptide | pGAPZ | 110 |
| Art v 1-02 | wild type signal peptide | pGAPZ | 40 |
| Art v 1-01 | α-factor signal peptide\textcircled{1} | pGAPZα | 200 |
| Art v 1-02 | α-factor signal peptide\textcircled{1} | pGAPZα | 70 |
| Art v 1-01 | α-factor signal peptide\textcircled{2} | pGAPZα | 210 |
| Art v 1-02 | α-factor signal peptide\textcircled{2} | pGAPZα | 30 |

Note:
\textcircled{1}The signal peptide was separated from the target protein by Ste 13signal cleavage sequence (amino acid sequence EAEA).
\textcircled{2}There was a Kex 2 enzyme cutting site between the signal peptide and the target protein, and there was no Ste 13 site and no other sequence.

### Example 6: Purification of Recombinant Art v 1 Protein

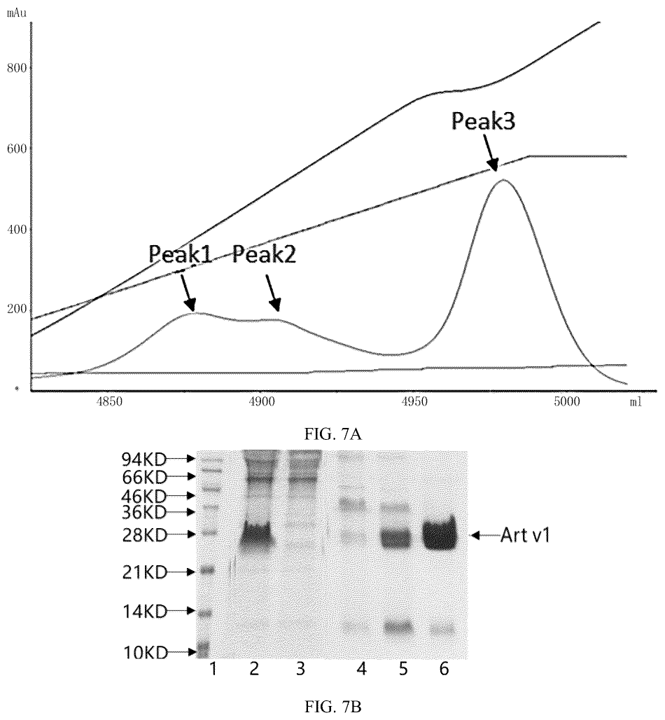

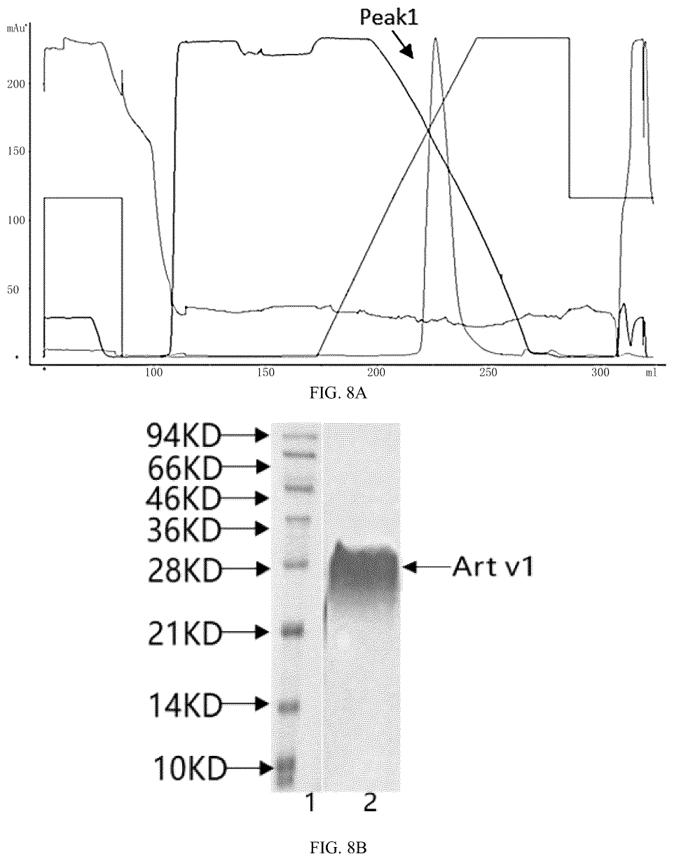

The expression clone selected in Example 5 was cultured at 1 liter using the method in Example 5, the fermentation broth was prepared, and the sample was purified by ion exchange and hydrophobic chromatography. The chromatographic packing was Hitrap SP HP, Hitrap Phenyl HP, and the specific steps were as follows:
1. Pretreatment of fermentation broth: centrifugated the fermentation broth at low temperature and high-speed, collected the supernatant, and concentrated the supernatant by 3KD membrane ultrafiltration, replaced to pH7.0 25 mM PB buffer, and filtered by 0.45 μm filter membrane.
2. Cation chromatography: the SP HP chromatography column was equilibrated with equilibrium buffer, and then the ultrafiltered fermentation liquid in the previous step was passed through the separation filler with purification system, and then the elution buffer was used for gradient elution, and the elution peak was collected; The equilibration buffer was 25 mM PB, pH7.0, and the elution buffer was 25 mM PB, 1.0M NaCl, pH7.0. As shown in FIGS. 7A-7B, the target protein was mainly in elution peak 3.
3. Hydrophobic chromatography: Art v 1 protein peaks collected in the previous step were diluted with equilibrium buffer, and Art v 1 protein solution was loaded on phenyl HP hydrophobic chromatography packing with equilibration buffer of 1.0M (NH₄)₂SO₄, 25 mM PB, pH7.0 and elution buffer of 25 mM PB, pH7.0. Collect elution peaks. As shown in FIGS. 8A-8B, there was only one elution peak with the target protein in the elution peak.
4. Ultrafiltration replacement: the protein peaks of hydrophobic chromatography were collected and replaced to pH7.0 25 mM PB.

After the above purification steps, the expression level of recombinant Art v 1 by pGAPZα-Art v 1-01 (with a Kex 2 enzyme cutting site between the signal peptide and the target protein, without Ste 13 site) was 94.5 mg/L, and the yield was 45%.

### Example 7: Purification of Native Art v 1 Protein

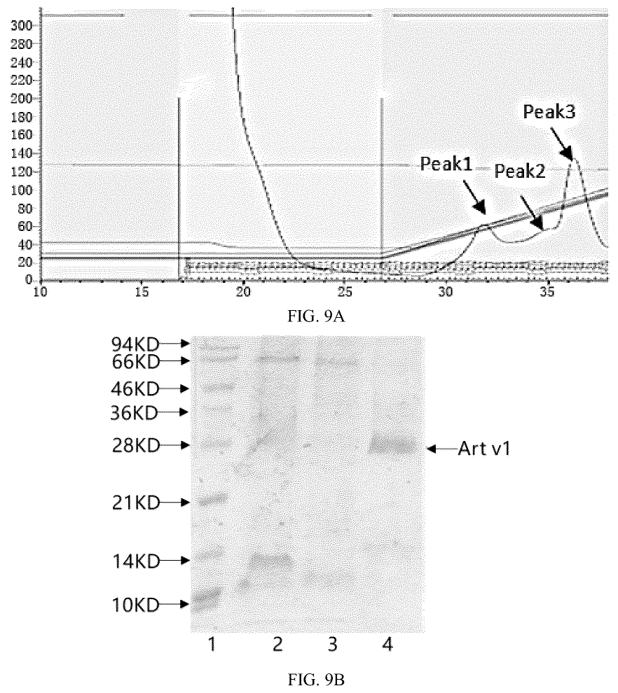

1. Preparation of crude extract: defatted Artemisia vulgaris pollen (purchased from Stallergenes Greer) was weighed. pH7.0, 50 mM PB solution was prepared, add PB solution at w/v ratio of 1:10, and extracted at low temperature for 48-72 hours; centrifugated at 4000 rpm and collected the supernatant to obtain the crude extract.
2. Chromatographic purification: The crude extract collected in step 2 was loaded to SP FF cation chromatography packing, the equilibrium buffer was 25 mM PB, pH7.0, and the elution buffer was 25 mM PB, 1.0M NaCl, pH7.0. The elution peak was collected and identified by electrophoresis. As shown in FIGS. 9A-9B, the native Art v 1 protein was predominantly in elution peak 3.
3. Ultrafiltration replacement: Merge elution peak 3 in step 2, concentrate the sample, replace to pH7.4 PBS solution, and freeze it below −20° C. until use.

### Example 8: Detection of the N Amino Acid Sequence and Molecular Weight of Art v 1 Protein by LC-MS

LC-MS molecular weight can accurately reflect whether the primary sequence of biological macromolecules is correct, including the N and C terminal sequences missing, and post-translational modifications such as glycosylation, oxidation and deamidation. It is one of the most important analytical methods for biological macromolecules. The molecular weight of purified recombinant Art v 1 protein with different construction methods was analyzed by LC-MS, and the results were shown in Table 2. When the α-factor signal peptide was used for secretion expression and there was Ste 13 enzyme cutting site between the signal peptide and the target gene, the corresponding amino acid sequence could not be completely removed. The resulting target protein had amino acid residues at the N-terminus. The N-terminal sequence of Art v 1 protein produced by the other construction form was completely consistent with the theory, and there was no residue.

TABLE 2
LC-MS molecular weights of recombinant Art v 1 protein expressed and
purified by different construction methods

| | | | The N-terminal first | Consistent with |
|---|---|---|---|---|
| Genetic | | Expression | five amino acids | the theoretical |
| sequence | Signal peptide | system | inferred by LC-MS | molecular weight |
| Art v 1-01 | wild type signal peptide | pPICZ | AGSKL | YES |
| Art v 1-02 | wild type signal peptide | pPICZ | AGSKL | YES |
| Art v 1-01 | α-factor signal peptide [symbol] | pPIC | AAGSKL | NO |
| Art v 1-02 | α-factor signal peptide [symbol] | pPICZα | AAGSKL | NO |
| Art v 1-01 | α-factor signal peptide [symbol] | pPICZα | AGSKL | YES |
| Art v 1-02 | α-factor signal peptide [symbol] | pPICZα | AGSKL | YES |
| Art v 1-01 | wild type signal peptide | pGAPZ | AGSKL | YES |
| Art v 1-02 | wild type signal peptide | pGAPZ | AGSKL | YES |
| Art v 1-01 | α-factor signal peptide [symbol] | pGAPZα | AAGSKL | NO |
| Art v 1-02 | α-factor signal peptide [symbol] | PGAPZα | LAAGSKL | NO |
| Art v 1-01 | α-factor signal peptide [symbol] | pGAPZα | AGSKL | YES |
| Art v 1-02 | α-factor signal peptide [symbol] | pGAPZα | AGSKL | YES |

Note: [symbol]
The signal peptide was separated from the target protein by Ste 13 signal cleavage sequence
(amino acid sequence EAEA). [symbol]
There was a Kex 2 enzyme cutting site between the signal peptide and the target protein, and
there was no Ste 13 site and no other sequence.

### Example 9: Peptide Mass Figerprinting of Art v 1 Protein

Peptide mass figerprinting is one of the most important identification methods in protein research. In theory, every protein has different peptides after digestion. The mass of these peptides is the peptide map of this protein. Alignment of the measured amino acid sequence with the known sequence allows one to know whether the amino acid primary structure of the analyzed protein is correct.

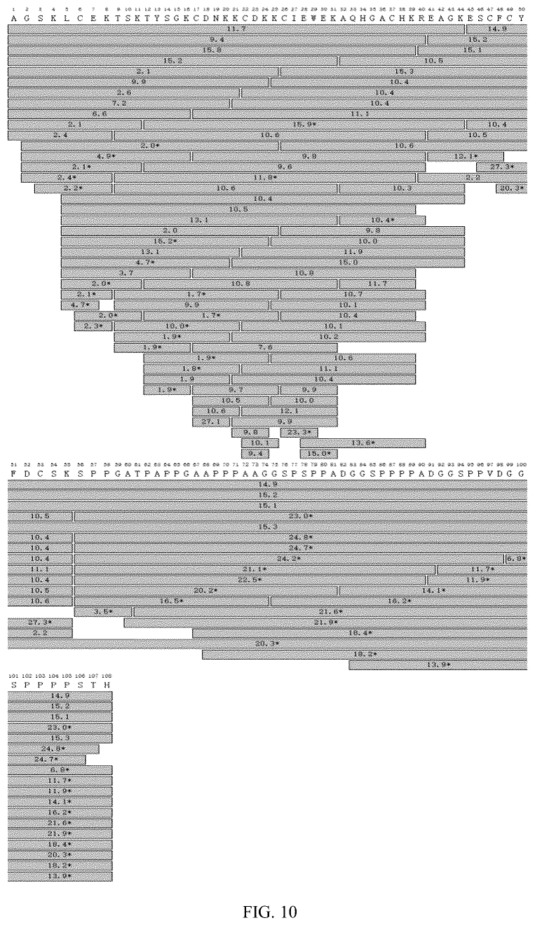

The purified Art v 1 expressed by strains with different construction methods in Example 8 was analyzed for peptide fragments. The results showed that except for the construction form with Ste 13 restriction site interval between α-factor and target gene, the coverage of recombinant Art v 1 protein obtained from other designed construction forms was 100% with the theoretical sequence. This indicated that the primary structure of Art v 1 protein was correct. FIG. 10 shows the detection results of peptide coverage of Art v 1 protein expressed by pGAPZ-Art v 1-01.

### Example 10: Disulfide Bond Detection of Art v 1 Protein

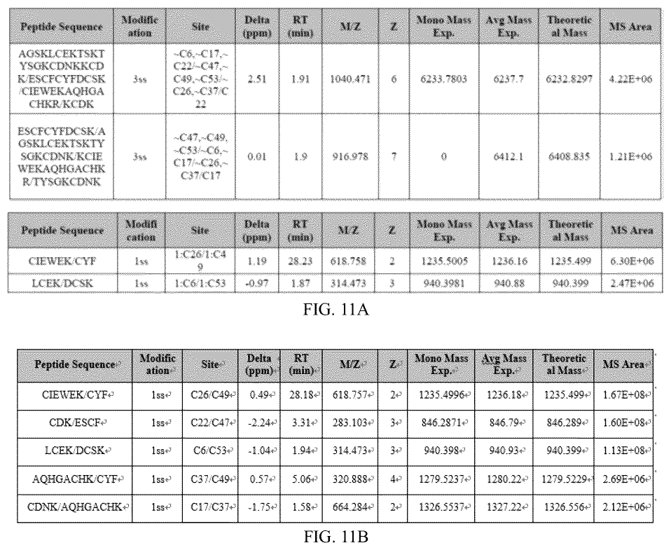

Whether disulfide bonds can be correctly paired is crucial for the maintenance of higher structure and activity of biological macromolecules such as proteins. The disulfide bond of native Art v 1 protein and recombinant Art v 1 protein obtained by pGAPZα-Art v 1-01 construction was determined by our Thermo Scientific Q Exactive LC-MS system, and the results were shown in FIGS. 11A-11B. FIG. 11A showed the identification of four theoretical disulfide bonds of recombinant Art v 1 protein C6/C53, C22/C47, C26/C49 and C17/C37 after trypsin and chymotrypsin double digestion. FIG. 11B showed the identification of four pairs of theoretical disulfide bonds of the native Art v 1 protein C6/C53, C22/C47, C26/C49, and C17/C37 after trypsin and chymotrypsin double digestion.

### Example 11: HPLC Determination of Art v 1 Protein Purity

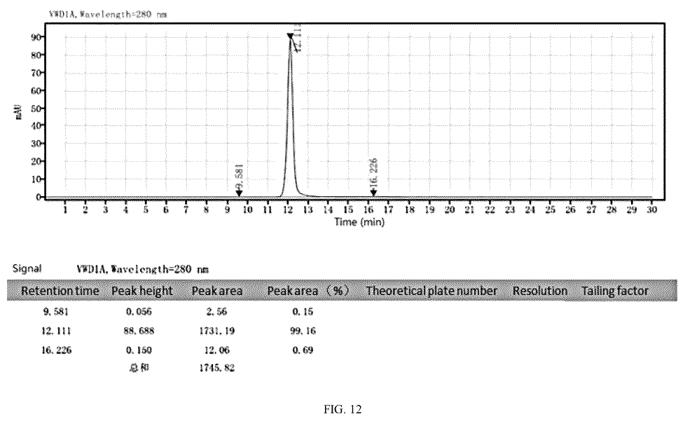

The purity of the purified samples was identified by electrophoresis: Agilient 1260 HPLC, column Sepax Zenix SEC-80, mobile phase 20 mM PB+300 mM NaCl (pH7.0) buffer, flow rate 0.5 ml/min, equal elution, column temperature 25.0° C., 280 nm to detect the purity of the samples. The results of FIG. 12 showed that the SEC-HPLC purity of recombinant Art v 1 protein obtained by pGAPZ-Art v 1-01 construction method was 99.16%, and the purity reached the pharmaceutical standard.

### Example 12: Activity Detection of Art v 1 Protein

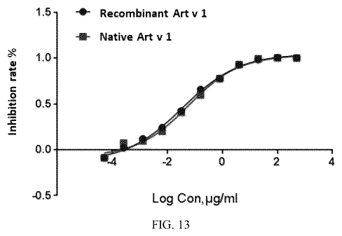

1. The recombinant Art v 1 protein obtained by pGAPZ-Art v 1-01 construction and the purified native Art v 1 protein prepared in Example 7 were diluted to 0.5 μg/ml with 50 mM NaH₂PO₄ buffer pH7.2, 100 μl per well, and coated at 4° C. overnight.
2. Sample preparation: the recombinant protein or native protein was diluted to the initial concentration of 500 μg/ml (S1) with blocking solution (2% BSA/PBST), and then diluted according to a 5-fold gradient, a total of 10 gradients (S2-S11), each dilution sample and positive serum (diluted 30 times) were mixed 1:1, then the samples were incubated at 4° C. overnight.
3. The next day, the ELISA plate was washed 3 times with PBST, then 200 μl of 2% BSA/PBST solution was added to each well and blocked for 2 hours at 37° C.
4. After blocking, the blocking solution was discarded, and the above mixed and incubated samples were added 100 μl per well and incubated for 1.5 h at 37° C. and 300 rpm.
5. After washing three times with PBST, secondary antibody of mouse anti-human IgE-HRP diluted 1:1500 was added 100 μl per well, incubated at 300 rpm for 1 h at 37° C.
6. Washed 3 times with PBST, and 100 μl TMB II color development solution was added to each well. After the reaction at 37° C. for 10 min, 50 μl termination solution (2M H₂SO₄) was added to each well, and OD450 nm was immediately detected.
7. Result analysis: As shown in FIG. 13, the inhibition curves of recombinant Art v 1 and native Art v 1 protein against IgE antibody in positive serum were basically the same. With native Art v 1 protein as the standard, the relative activity of recombinant Art v 1 was 118.7%. The results showed that the recombinant protein had similar immune response activity in vitro with IgE specific antibody in serum of allergic patients.

What is claimed is:

1. A protein for treating an Artemisia pollen allergy, wherein the protein is a recombinant type I allergen protein of Artemisia vulgaris pollen (Art v 1), and the recombinant Art v 1 has an amino acid sequence, a disulfide bond, and a molecular weight being consistent with a native Art v 1, and an immune response activity of the recombinant Art v 1 in vitro with a specific antibody in a serum of an allergic patient is equivalent to an immune response activity of the native Art v 1 in vitro with the specific antibody in the serum of the allergic patient.

2. The protein for treating the Artemisia pollen allergy according to claim 1, wherein the amino acid sequence is set forth in SEQ ID NO: 4.

3. A polynucleotide encoding the protein for treating the Artemisia pollen allergy according to claim 2, wherein the polynucleotide has the base sequence as set forth in SEQ ID NO: 13.

4. A vector comprising the polynucleotide encoding the protein for treating the Artemisia pollen allergy according to claim 3, wherein the vector is selected from the group consisting of pAO815, pPIC9, pPIC9K, pPIC3.5, pPIC3.5K, pPICZαA, pPICZαB, pPICZαC, pGAPZαA, pGAPZαB, pGAPZαC, pPICZ A, pPICZ B, pPICZ C, pGAPZ A, pGAPZ B, and pGAPZ C.

5. A Pichia pastoris strain comprising the vector according to claim 4, wherein the Pichia pastoris strain is selected from the group consisting of SMD1168, GS115, KM71, X33, and KM71H.

6. An expression method of the protein for treating the Artemisia pollen allergy according to claim 1, comprising the following steps:
step A: constructing a vector comprising a gene sequence encoding the recombinant Art v 1, wherein the vector comprises a polynucleotide encoding the protein for treating the Artemisia pollen allergy, the polynucleotide has the base sequence as set forth in SEQ ID NO: 13, the recombinant Art v 1 has the amino acid sequence set forth in SEQ ID NO: 4, the vector is selected from the group consisting of pAO815, pPIC9, pPIC9K, pPIC3.5, pPIC3.5K, pPICZαA, pPICZαB, pPICZαC, pGAPZαA, pGAPZαB, pGAPZαC, pPICZ A, pPICZ B, pPICZ C, pGAPZ A, pGAPZ B, and pGAPZ C:
when a wild-type signal peptide is used for a secretory expression, cloning the gene sequence encoding the recombinant Art v 1 with a wild-type signal peptide downstream of a promoter and upstream of a terminator in the vector to construct an expression cassette, wherein the gene sequence is designed to comprise a start codon and a stop codon; or
when an α-factor signal peptide in the vector is used for an expression, cloning the gene sequence encoding the recombinant Art v 1 downstream of a sequence encoding a Kex2 signal peptide cleavage site having an amino acid sequence of KR in the vector, so that after the expression, the Kex2 signal peptide cleavage site exists between the α-factor signal peptide and the recombinant Art v 1 as a target protein, and no Ste 13 site having the amino acid sequence of EAEA as set forth in SEQ ID NO: 15 or other sequence exists between the α-factor signal peptide and the recombinant Art v 1 as the target protein, wherein the gene sequence encoding the recombinant Art v 1 does not comprise a start codon and is designed to comprise a stop codon;
step B: linearizing the vector constructed in the step A to obtain a linearized vector, transforming the linearized vector into a Pichia pastoris strain to obtain a transformed Pichia pastoris strain, and culturing the transformed Pichia pastoris strain at suitable conditions to produce a fermentation broth comprising the target protein; and
step C: recovering and purifying the target protein from the fermentation broth.

7. A purification method of the protein for treating the Artemisia pollen allergy according to claim 1, comprising the following steps:
step A: centrifugating a fermentation broth comprising the recombinant Art v 1 at a low temperature and a high speed to obtain a supernatant, collecting the supernatant and subjecting the supernatant to a concentration by an ultrafiltration with a 3 KD membrane package and a buffer exchange with a 25 mM phosphate buffer (PB) at pH 7.0, followed by a filtration by a 0.45 μm filter membrane to obtain a treated fermentation supernatant;
step B: cation chromatography: equilibrating a chromatography column with a first equilibration buffer, and then allowing the treated fermentation supernatant obtained in the step A to pass through a separation packing by a purification system, followed by a gradient elution by a first elution buffer, and collecting a first elution peak to obtain a recombinant Art v 1 protein peak, wherein the first equilibration buffer is the 25 mM PB at pH 7.0, and the first elution buffer is a 25 mM PB/1.0 M NaCl at pH 7.0;
step C: hydrophobic interaction chromatography: diluting the recombinant Art v 1 protein peak obtained in the step B with a second equilibration buffer to obtain a diluted recombinant Art v 1 protein solution, and equilibrating the chromatography column with the second equilibration buffer, loading the diluted recombinant Art v 1 protein solution onto a hydrophobic interaction chromatography packing, followed by an elution by a second elution buffer, and collecting a second elution peak to obtain a target protein peak, wherein the second equilibration buffer is a 1.0 M (NH₄)₂SO₄/25 mM PB at pH 7.0, and the second elution buffer is the 25 mM PB at pH 7.0; and
step D: ultrafiltration and buffer exchange: subjecting the target protein peak obtained in the step C to the ultrafiltration and the buffer exchange with a buffer being the 25 mM PB at pH 7.0, followed by the filtration and a bacteria removal to obtain a recombinant Art v 1 protein stock solution.

8. A method for treating an Artemisia pollen allergy, comprising administering to a subject a drug comprising the protein according to claim 1.

9. A method for detecting an Artemisia pollen allergy, comprising administering to a subject a diagnostic reagent comprising the protein according to claim 1.

10. The purification method according to claim 7, wherein the amino acid sequence of the recombinant Art v 1 is set forth in SEQ ID NO: 4.

11. The method according to claim 8, wherein the amino acid sequence of the recombinant Art v 1 is set forth in SEQ ID NO: 4.

12. The method according to claim 9, wherein the amino acid sequence of the recombinant Art v 1 is set forth in SEQ ID NO: 4.


SEQUENCE LISTING (16 sequences)

SEQ ID NO: 1 - DNA, 396 bp, Artemisia vulgaris
  source 1..396: mol_type = other DNA; note = Unoptimized DNA sequence encoding native Art v 1 with wild-type signal peptide
atggcaaagtgttcatatgttttctgtgcggttcttctgattttcatagttgctatcggagaaatggaggccgctggttcaaagttgtgtgaaaagacaagcaagacgtattcgggtaagtgcgacaacaagaaatgtgacaaaaagtgtatagagtgggagaaagcgcaacatggtgcttgtcacaagagagaagccggcaaagaaagttgcttttgctactttgactgttccaaatcgcctcctggagcaacaccagcgcctcctggtgcagctcctcccccagctgctggcggctctccgtcacctcccgctgatggtggctcaccacctcctccagctgatggtggatctcctcctgtagatggtggctctccacctcctccgtccactcac

SEQ ID NO: 2 - DNA, 396 bp, Artemisia vulgaris
  source 1..396: mol_type = other DNA; note = Nucleotide sequence of Art v 1-01 obtained after codon optimization of DNA sequence encoding native Art v 1 with wild-type signal peptide
atggcaaaatgttcttacgttttctgtgcggttttgctgattttcattgttgctattggtgaaatggaagctgctggttctaagttgtgtgagaagactagcaagacttactcgggtaagtgcgacaacaagaagtgtgacaagaagtgtattgaatgggagaaagcgcaacatggtgcttgtcacaagagagaggctggtaaagaaagttgcttctgctacttcgactgttccaactcgcctcctggagcaactccagcgccacctggagcagctcctccaccagctgctggcggctctccgtcacctccagctgatggaggttcaccacctccaccagctgatggtggatctccacctgttgatggtggatctccacctccaccatctactcat

SEQ ID NO: 3 - DNA, 396 bp, Artemisia vulgaris
  source 1..396: mol_type = other DNA; note = Nucleotide sequence of Art v 1-02 obtained after codon optimization of DNA sequence encoding native Art v 1 with wild-type signal peptide
atggcaaagtgttcatatgttttctgtgcggttcttctgattttcatagttgctatcggagaaatggaggccgctggttctaaattgtgtgaaaaaacttctaagacctactctggtaaatgtgataacaagaagtgtgataaaaagtgtatcgaatgggaaaaagctcaacatggtgcttgtcataaaagagaagctggtaaagaatcttgcttctgttacttcgattgttctaagtctccaccaggtgctactcctgctcctccaggtgctgctccacctccagctgctggtggttctccatctccaccagctgatggtggttctcctccaccaccagctgacggtggttctccccctgttgatggtggttccccaccaccaccttctactcat

SEQ ID NO: 4 - protein, 108 aa, Artemisia vulgaris
  source 1..108: note = Amino acid sequence of Art v 1 protein
AGSKLCEKTSKTYSGKCDNKKCDKKCIEWEKAQHGACHKREAGKESCFCYFDCSKSPPGATPAPPGAAPPPAAGGSPSPPADGGSPPPPADGGSPPVDGGSPPPPSTH

SEQ ID NO: 5 - DNA, 21 bp, Synthetic construct
  source 1..21: mol_type = other DNA; note = Forward primer 5'AOX primer
gactggttccaattgacaagc

SEQ ID NO: 6 - DNA, 21 bp, Synthetic construct
  source 1..21: mol_type = other DNA; note = Reverse primer 3'AOX primer
gcaaatggcattctgacatcc

SEQ ID NO: 7 - DNA, 29 bp, Synthetic construct
  source 1..29: mol_type = other DNA; note = Forward primer for Art v 1-01 without signal peptide
ccgctcgagaaaagagctggttcaaagtt

SEQ ID NO: 8 - DNA, 29 bp, Synthetic construct
  source 1..29: mol_type = other DNA; note = Reverse primer for Art v 1-01 without signal peptide
gctctagattatcagtgagtggacggagg

SEQ ID NO: 9 - DNA, 29 bp, Synthetic construct
  source 1..29: mol_type = other DNA; note = Forward primer for Art v 1-02 without signal peptide
ccgctcgagaaaagagctggttctaaatt

SEQ ID NO: 10 - DNA, 29 bp, Synthetic construct
  source 1..29: mol_type = other DNA; note = Reverse primer for Art v 1-02 without signal peptide
gctctagattatcaatgagtagaaggtgg

SEQ ID NO: 11 - protein, 89 aa, Synthetic construct
  source 1..89: note = a-factor signal peptide
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 12 - protein, 24 aa, Synthetic construct
  source 1..24: note = Wild-type signal peptide
MAKCSYVFCAVLLIFIVAIGEMEA

SEQ ID NO: 13 - DNA, 324 bp, Artemisia vulgaris
  source 1..324: mol_type = other DNA; note = Art v 1-01 without signal peptide
gctggttctaagttgtgtgagaagactagcaagacttactcgggtaagtgcgacaacaagaagtgtgacaagaagtgtattgaatgggagaaagcgcaacatggtgcttgtcacaagagagaggctggtaaagaaagttgcttctgctacttcgactgttccaactcgcctcctggagcaactccagcgccacctggagcagctcctccaccagctgctggcggctctccgtcacctccagctgatggaggttcaccacctccaccagctgatggtggatctccacctgttgatggtggatctccacctccaccatctactcat

SEQ ID NO: 14 - DNA, 324 bp, Artemisia vulgaris
  source 1..324: mol_type = other DNA; note = Art v 1-02 without signal peptide
gctggttctaaattgtgtgaaaaaacttctaagacctactctggtaaatgtgataacaagaagtgtgataaaaagtgtatcgaatgggaaaaagctcaacatggtgcttgtcataaaagagaagctggtaaagaatcttgcttctgttacttcgattgttctaagtctccaccaggtgctactcctgctcctccaggtgctgctccacctccagctgctggtggttctccatctccaccagctgatggtggttctcctccaccaccagctgacggtggttctccccctgttgatggtggttccccaccaccaccttctactcat

SEQ ID NO: 15 - protein, 4 aa, Synthetic construct
  source 1..4: note = Amino acid sequence of Ste 13 site
EAEA

SEQ ID NO: 16 - DNA, 396 bp, Artemisia vulgaris
  source 1..396: mol_type = other DNA; note = Consensus sequence of unoptimized DNA sequence encoding native Art v 1 with wild-type signal peptide, Art v 1-01, and Art v 1-02
atggcaaartgttcwtaygttttctgtgcggttytkctgattttcatwgttgctatyggwgaaatggargcygctggttcwaarttgtgtgaraaracwwsyaagacbtaytckggtaartgygayaacaagaartgtgayaaraagtgtathgartgggaraaagckcaacatggtgcttgtcayaaragagargcyggyaaagaawsttgcttytgytacttygaytgttcyaavtckccwccwggwgcwacwccwgckccwccwggwgcwgctccwcchccagctgctggyggytctccrtcwccwccmgctgatggwggytcwccwccwccwccagctgayggtggwtctcchcctgtwgatggtgghtcyccaccwccwccdtcyactcay